(19) United States
(12) Patent Application Publication
(10) Pub. No.: US 2021/0100830 A1
(43) Pub. Date: Apr. 8, 2021

(54) STARCH-BASED HEMOSTATIC POWDER AND PREPARATION METHOD THEREOF

(71) Applicant: QINGDAO CHENLAND MARINE BIOTECHNOLOGY CO., LTD., Qingdao (CN)

(72) Inventors: Baoqun WANG, Qingdao (CN); Shasha LIN, Qingdao (CN); Shengcan ZOU, Qingdao (CN)

(21) Appl. No.: 17/125,618

(22) Filed: Dec. 17, 2020

Related U.S. Application Data
(63) Continuation of application No. PCT/CN2019/090099, filed on Jun. 5, 2019.

(30) Foreign Application Priority Data
Jul. 25, 2018 (CN) 201810822800.X

(51) Int. Cl. A61K 31/718; C08B 30/12; A61K 9/14; A61P 31/04; A61P 17/02

(52) U.S. Cl. CPC A61K 31/718; C08B 30/12; A61P 17/02; A61P 31/04; A61K 9/14

(57) ABSTRACT

Disclosed is a preparation method of a starch-based hemostatic powder, comprising the following steps: (1) starch modification; (2) emulsification and crosslinking; (3) separation, purification, drying and sterilization. Provided is a starch-based hemostatic powder, which has a simple preparation process and a controllable cost. After being modified, raw material medical potato starch has a high degree of emulsification and crosslinking and a good and uniform spherical shape, and has a porous structure. Moreover, the product has good biocompatibility, low biological cytotoxicity, and obvious antibacterial effect; the product has a high liquid absorption ratio, and quickly forms a gel to cover the wound surface after liquid absorption, thus achieving a good hemostatic effect. The starch-based hemostatic powder is an excellent biomedical product, and can be used for clinical wound treatment, surgery to prevent organ adhesion, and bleeding from various wounds.

### BACKGROUND OF THE INVENTION

### 1. Technical Field

The present invention belongs to the field of medical supplies, and particularly relates to a medical hemostatic powder using modified potato starch as a raw material and the preparation method thereof.

### 2. Description of Related Art

In traumatic injuries or surgical operations, quick and effective hemostasis is of great significance to saving lives. At present, absorbable hemostatic materials, especially novel in vivo absorbable hemostatic materials developed in recent years, can not only coagulate blood in a very short time to achieve the purpose of rapid hemostasis, but also can be absorbed by the body in a short time. Common absorbable hemostatic materials include collagen and microfibrous collagen, medical hemostatic gelatin, oxidized cellulose and oxidized regenerated cellulose, cyanoacrylic tissue glue, and fibrin hemostatic materials. These materials achieved good results in animal experiments and clinical applications, but they also have some shortcomings: collagen and gelatin are raw materials derived from animal tissues and are heterogeneous proteins prone to rejection and have potential sensitization, and due to slow absorption rate, they will increase the risk of wound infection; for celluloses, the human body lacks enzymes to make them degrade, and it takes a long time to degrade celluloses, which may bring side effects such as infection to patients.

As a plant polysaccharide, starch is widely available and has low price and good biocompatibility and degradability. In recent years, the development of new products or new application methods using starch as a raw material has become a hot research topic. However, the current starch-based hemostatic material has a simple function and does not have an antibacterial effect. In addition, the mechanism of hemostasis is simple, resulting in a hemostatic effect inferior to chitosan and other hemostatic materials.

In the prior art, methods for preparing a variety of polysaccharide hemostatic powders have been disclosed, including concentrated specific modified starches, such as cationic starch, epichlorohydrin cross-linked starch, carboxymethyl starch, hydroxyethyl starch, and pregelatinized modified starch, pregelatinized hydroxypropyl distarch phosphate, acrylic acid-carboxymethyl starch graft copolymer, etc. As a hemostatic material, cationic starch is used to attract negatively charged red blood cells due to the positive charge on its surface, and interact with the negatively charged red blood cells, thereby accelerating the process of blood clotting; on the other hand, after the positively charged modified starch is in contact with blood, it can tightly adhere to the tissue, seal the wound, and quickly stop bleeding.

Du Baotang et al., processed raw potato starch through gelatinization, enzymatic hydrolysis, emulsification and crosslinking to prepare MMPH porous microspheres. The process is more complicated, and the prepared starch microspheres have a slower water absorption rate and poor adhesion, and thus have poor hemostatic effect for large-area bleeding (“Medical Equipment”, 2014, 35(3):23-25).

As disclosed in Chinese patent CN201510171482.1, vegetable starch and etherified starch were subjected to radiation treatment to obtain two starches with better reactivity, the two starches were mixed to have emulsification and crosslinking reaction, and the reaction product was subjected to extraction and washing to obtain starch-based hemostatic powder. γ-ray irradiation is complicated to the starch modification process. By controlling different irradiation doses, modified starches with different grafting activities and different polymer molecular weights can be produced. Pure starch will be degraded by ⁶⁰Coγ-ray irradiation to the accompaniment of processes such as oxidation and hydrolysis. Irradiation technology has obvious limitations. Special equipment is required to provide radiation sources and safety protection measures are required, which increases production costs greatly; and due to changes in sensory properties under high radiation doses, appropriate radiation doses need to be controlled; moreover, due to the differences in the history, living habits and laws and regulations of various countries, the types of medical supplies that are allowed to conduct irradiation are quite different in the world.

Chinese patents CN201710072673.1 and CN201610865770.1 respectively disclose a composite starch-based hemostatic powder and a composite microporous cross-linked starch-based hemostatic powder, where sodium alginate, sodium carboxymethyl cellulose and potato starch are compounded to obtain raw material, sodium alginate and sodium carboxymethyl cellulose can form dimers and form confluence points with other chains, and finally form a gel network, so that potato starch forms a microporous structure and the water absorption of the entire starch-based hemostatic agent is enhanced to improve the hemostatic effect of potato starch used alone. However, since the gel formed by sodium alginate without ionic crosslinking is reversible in the solution, the hemostatic particles formed by virtue of an emulsifier have poor spherical integrity and uneven dispersion, and adhesion between the particles may affect the formation of the particles.

In view of the above reasons, it is desired to overcome the above-mentioned problems of the prior art and provide a hemostatic powder with excellent hemostatic effect, good biocompatibility and antibacterial effect.

### BRIEF SUMMARY OF THE INVENTION

In view of the problems in the prior art, the objective of the present invention is to provide a starch-based hemostatic powder and a preparation method thereof. The prepared hemostatic powder has good hemostatic effect, good biocompatibility, and no antigenic reaction, and thus is suitable for hemostasis of bleeding, arteriovenous hemorrhage and hard-to-reach area bleeding during operation.

In order to achieve the above objective, the present invention adopts the following technical solutions:

A preparation method of a starch-based hemostatic powder, including the following steps:

(1) gelatinizing and modifying potato starch raw material to obtain modified starch;

(2) emulsifying and crosslinking the modified starch, obtained in Step (1), in an emulsifier, thus obtaining cross-linked starch; and

(3) carrying out separation, purification, drying and sterilization on the cross-linked starch obtained in Step (2), thus obtaining the starch-based hemostatic powder.

In Step (1), the mass concentration of starch is between 1% and 20%, the gelatinization temperature is between 40° C. and 80° C., and the gelatinization time is between 20 minutes and 120 minutes; after the gelatinization treatment, the pH is adjusted to a range from 8 to 12.

In Step (1), a starch modifier is 2-chloroethyldiethylamine with a mass concentration between 1% and 10%, and the reaction time is between 5 h and 24 h.

In Step (2), the emulsifier is one or more of Tween-60, Tween-80, Span-60, and Span-80.

In the above Step (2), the emulsifier is first dissolved in an oil phase, heated and stirred uniformly for 10-50 minutes, and then the modified starch obtained in Step (1) is added dropwise to the solution.

The oil phase is one of liquid paraffin, aviation kerosene, and vegetable oil; the emulsifier accounts for 0.1%-10% of the oil phase in a mass ratio, and the volume ratio of the oil phase to the aqueous solution of the starch is 3-6:1.

In Step (2), a crosslinking agent is epichlorohydrin, and the crosslinking agent accounts for 0.2%-10% of the oil phase in a mass ratio.

In Step (2), the modified starch is emulsified for 0.5-12 h at a temperature between 35° C. and 80° C., the stirring speed of the emulsification and crosslinking process is between 100 rpm/min and 1000 rpm/min, and the crosslinking time is between 1 h and 72 h.

The present invention discloses a starch-based hemostatic powder prepared by the above method.

The present invention also provides the application of the starch-based hemostatic powder to provide one effect of hemostatic, anti-adhesion, antibacterial, and wound sealing effects on bloody wounds or their combinations; the blooding wound refers to the body surface, tissues and organs in the body, or tissues in the body cavity or organs in the body cavity of mammals, birds, and reptiles.

Due to the application of the above technical solution, the present invention designs a starch-based hemostatic powder with the following advantages:

According to the present invention, the natural starch is modified to simulate the mechanism of microporous polysaccharide hemostasis; through the starch modification, specific groups are introduced on the molecular chain of the starch so that the starch has better hemostatic and antibacterial effects than ordinary microporous polysaccharides.

The starch-based hemostatic powder provided by the present invention has a simple preparation process and a controllable cost. Medical potato starch is used as a raw material and undergoes a modification treatment to realize a high degree of emulsification and crosslinking and good and uniform spherical shape. The particle size of the starch-based hemostatic powder microspheres is between 20 μm and 180 μm, and the microspheres with a particle size between 50 μm and 150 μm account for no less than 70% of the total microsphere particles; and the starch-based hemostatic powder has a porous structure. Moreover, the hemostatic powder disclosed in the present invention has good biocompatibility, high liquid absorption multiple, fast water absorption, and quickly forms a gel to cover the wound surface after liquid absorption. With good hemostatic effect and excellent antibacterial performance, the hemostatic powder is an excellent biomedical product.

The hemostatic powder provided by the invention has low toxicity to biological cells and obvious antibacterial effect, and can be used for clinical wound treatment, surgery to prevent organ adhesion, and various wound bleeding.

### BRIEF DESCRIPTION OF THE SEVERAL VIEWS OF THE DRAWINGS

FIG. 1 is a scanning electron micrograph of the starch-based hemostatic powder prepared in Example 2;

FIG. 2 is an in vitro hemostatic test of the starch-based hemostatic powder prepared in Example 2; and

FIG. 3 is a skin irritation test of the starch-based hemostatic powder prepared in Example 3.

### DETAILED DESCRIPTION OF THE INVENTION

The above-mentioned contents of the present invention will be further described in detail through the specific implementation solutions provided in the following embodiments. Those skilled in the art should not understand that the scope of the above-mentioned subject matter of the present invention is limited to the following embodiments. Any technology implemented on the basis of the above-mentioned contents of the present invention belongs to the scope of the present invention.

The experimental methods adopted in the following embodiments, unless otherwise specified, are conventional methods; the reagents, materials, instruments and the like used in the following embodiments, if not specifically stated, are commercially available.

### Example 1

(1) Gelatinization of starch: 10 g of potato starch was weighed and dissolved in 600 mL of purified water, and then heated and stirred to be gelatinized at 75° C. for 30 min; the resulting solution was stirred uniformly and adjusted with a sodium hydroxide solution until the pH reached a range from 8 to 12, thus obtaining a gelatinized starch solution.

(2) Modification of starch: 1 g of 2-chloroethyldiethylamine was weighed and dissolved in 10 mL of water, the resulting solution was added to the gelatinized starch solution to further react at 70° C. for 5 h, thus obtaining a modified starch solution.

(3) Preparation of oil phase: 500 mL of liquid paraffin was weighed and, added with 2 g of Tween-80, and the resulting material was stirred at 50° C. for 10 min and set aside for later use.

(4) Emulsification and crosslinking: the modified starch was dropwise added to the oil phase and stirred to react at 70° C. for 40 min, 10 mL of epichlorohydrin was added to carry out crosslinking reaction for 2 h, and the reaction solution was set still after the reaction.

(5) Separation of precipitation: after the reaction solution was set still, the upper flowing liquid was removed and the lower milky white liquid was centrifuged and separated.

(6) Purification and drying: the milky body after centrifugation was washed with absolute ethanol and then subjected to suction filtration and vacuum drying at 40° C. The dried powder was sieved with a screen to obtain the product before packaging.

(7) Packaging and sterilization: the sieved product was packaged in polyethylene bottles and then plastic-sealed, and then sterilized.

### Example 2

(1) Gelatinization of starch: 10 g of potato starch was weighed and dissolved in 800 mL of purified water, and then heated and stirred to be gelatinized at 50° C. for 60 min; the resulting solution was stirred uniformly and adjusted with a sodium hydroxide solution until the pH reached a range from 8 to 12, thus obtaining a gelatinized starch solution.

(2) Modification of starch: 3 g of 2-chloroethyldiethylamine was weighed and dissolved in 50 mL of water, the resulting solution was added to the gelatinized starch solution to further react at 70° C. for 10 h, thus obtaining a modified starch solution.

(3) Preparation of oil phase: 1200 mL of aviation kerosene was weighed and, added with 10 g of Tween-80 and 10 g of Span-80, and the resulting material was stirred at 60° C. for 30 min and set aside for later use.

(4) Emulsification and crosslinking: the modified starch was dropwise added to the oil phase and stirred to react at 60° C. for 60 min, 50 mL of epichlorohydrin was added to carry out crosslinking reaction for 5 h, and the reaction solution was set still after the reaction.

(5) Separation of precipitation: after the reaction solution was set still, the upper flowing liquid was removed and the lower milky white liquid was centrifuged and separated.

(6) Purification and drying: the milky body after centrifugation was washed with absolute ethanol and then subjected to suction filtration and vacuum drying at 40° C. The dried powder was sieved with a screen to obtain the product before packaging.

(7) Packaging and sterilization: the sieved product was packaged in polyethylene bottles and then plastic-sealed, and then sterilized.

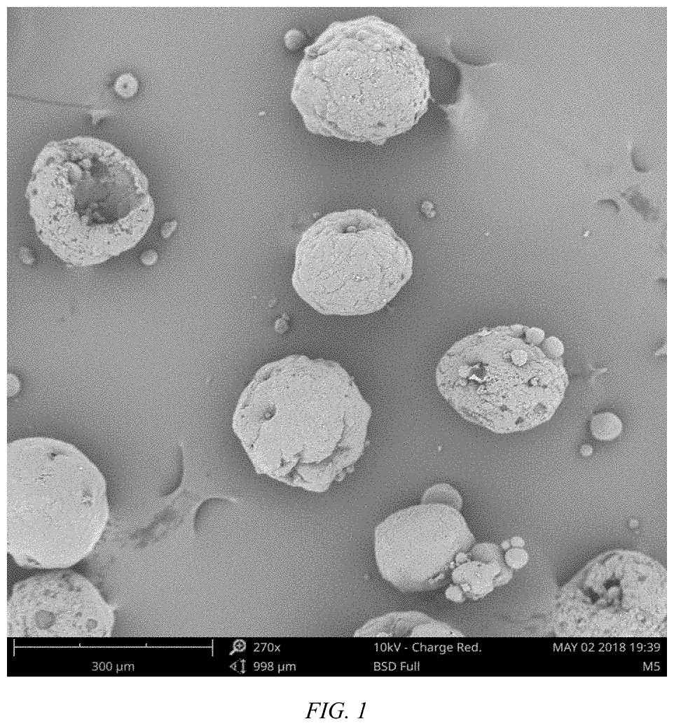

The obtained starch-based hemostatic powder was photographed by a scanning electron microscope, as shown in FIG. 1. The particle size of the starch-based hemostatic powder microspheres is between 20 μm and 180 μm, and the microspheres with a particle size between 50 μm and 150 μm account for not less than 70% of the total microsphere particles.

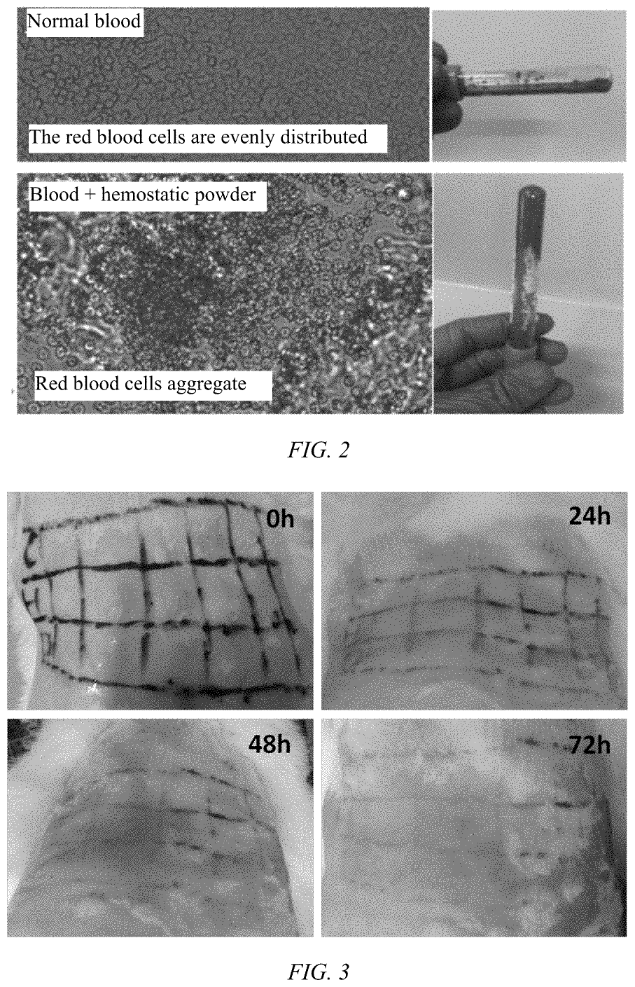

The obtained starch-based hemostatic powder was tested for hemostasis, and the hemostatic effect is shown in FIG. 2.

2 mL of rabbit blood was taken and placed in a 10 mL test tube, 0.5 mg of heparin sodium was added, and after being mixed, the rabbit blood and heparin took a long-lasting anticoagulant effect and achieved good blood fluidity. Then, 50 mg of the above hemostatic powder was taken and added to the rabbit blood, and the rabbit blood was then shaken gently for 5-10 seconds. It was observed that a large number of blood clots were formed, indicating that the coagulation mechanism was quickly activated. Under a microscope, as shown in the photomicrograph in the left image of FIG. 2, red blood cells in normal blood are evenly distributed without aggregation; however, the red blood cells of blood added with the hemostatic powder aggregate obviously, the local concentration increases, and the activation of coagulation process is accelerated.

### Example 3

(1) Gelatinization of starch: 15 g of potato starch was weighed and dissolved in 600 mL of purified water, and then heated and stirred to be gelatinized at 80° C. for 20 min; the resulting solution was stirred uniformly and adjusted with a sodium hydroxide solution until the pH reached a range from 8 to 12, thus obtaining a gelatinized starch solution.

(2) Modification of starch: 4 g of 2-chloroethyldiethylamine was weighed and dissolved in 50 mL of water, the resulting solution was added to the gelatinized starch solution to further react at 70° C. for 5 h, thus obtaining a modified starch solution.

(3) Preparation of oil phase: 1800 mL of liquid paraffin was weighed and, added with 4 g of Tween-80 and 4 g of Span-80, and the resulting material was stirred at 60° C. for 30 min and set aside for later use.

(4) Emulsification and crosslinking: the modified starch was dropwise added to the oil phase and stirred to react at 60° C. for 60 min, 50 mL of epichlorohydrin was added to carry out crosslinking reaction for 5 h, and the reaction solution was set still after the reaction.

(5) Separation of precipitation: after the reaction solution was set still, the upper flowing liquid was removed and the lower milky white liquid was centrifuged and separated.

(6) Purification and drying: the milky body after centrifugation was washed with absolute ethanol and then subjected to suction filtration and vacuum drying at 40° C. The dried powder was sieved with a screen to obtain the product before packaging.

(7) Packaging and sterilization: the sieved product was packaged in polyethylene bottles and then plastic-sealed, and then sterilized.

### Comparative Example 1

(1) Gelatinization of starch: 10 g of potato starch was weighed and dissolved in 500 mL of purified water, and then stirred to be gelatinized; 5 g of sodium carboxymethyl cellulose was added, and the pH of the resulting solution was adjusted to 11 with a sodium hydroxide solution.

(2) Preparation of oil phase: 500 mL of vegetable oil was added to a reaction kettle and heated to 70° C., and a mixture containing 10 g of a mixture of Span-80 and 10 g of Tween-80 was added.

(3) Emulsification and crosslinking: the gelatinized starch was mixed with the above oil phase, and 20 mL of epichlorohydrin was then added to react for 24 h, and the resulting material was taken.

(4) Separation of precipitation: absolute ethanol and ethyl acetate were added to separate the resulting material, and the upper oil phase was poured out.

(5) Purification and drying: the milky body after centrifugation was washed with absolute ethanol and then subjected to suction filtration and vacuum drying at 40° C. The dried powder was sieved with a screen to obtain the product before packaging.

(6) Packaging and sterilization: the sieved product was packaged in polyethylene bottles and then plastic-sealed, and then sterilized.

### Comparative Example 2

(1) Gelatinization of starch: 10 g of potato starch was weighed and dissolved in 800 mL of purified water, and then heated and stirred to be gelatinized at 50° C. for 60 min; the resulting solution was stirred uniformly and adjusted with a sodium hydroxide solution until the pH reached a range from 8 to 12, thus obtaining a gelatinized starch solution.

(2) Modification of starch: 3 g of 2-chloroethyldiethylamine was weighed and dissolved in 50 mL of water, the resulting solution was added to the gelatinized starch solution to further react at 70° C. for 10 h, thus obtaining a modified starch solution.

(3) Preparation of oil phase: 1200 mL of aviation kerosene was weighed and, added with 10 g of Tween-80 and 10 g of Span-80, and the resulting material was stirred at 60° C. for 30 min and set aside for later use.

(4) Emulsification and crosslinking: the modified starch was dropwise added to the oil phase and stirred to react at 60° C. for 60 min, 50 mL of epichlorohydrin was added to carry out crosslinking reaction for 5 h, and the reaction solution was set still after the reaction.

(5) Esterification modification: 5 mL of sodium trimetaphosphate solution was added to the modified starch after crosslinking, and the resulting solution was stirred to react at 35° C. to prepare a hydroxypropyl distarch phosphate solution;

(6) Ultrasonic treatment: the hydroxypropyl distarch phosphate solution (ice bath) was placed in an ultrasonic cell pulverizer and treated at a power of 450 w for 3 min (running for 2 s with a stop of 2 s).

(7) Separation of precipitation: after the reaction solution was set still, the upper flowing liquid was removed and the lower milky white liquid was centrifuged and separated.

(8) Purification and drying: the milky body after centrifugation was washed with absolute ethanol and then subjected to suction filtration and vacuum drying at 40° C. The dried powder was sieved with a screen to obtain the product before packaging.

(9) Packaging and sterilization: the sieved product was packaged in polyethylene bottles and then plastic-sealed, and then sterilized.

### Comparative Example 3

(1) Enzymatic hydrolysis of starch: 100 g of potato starch and 10 g of amylase were added in 250 mL of phosphate buffer (pH=5), the resulting solution was stirred evenly to take a hydrolysis reaction at 45° C. for 8 h, the reaction solution was subjected to centrifugal separation at a rotating speed of 4000 r/min, the precipitation was then dried in vacuum to obtain hydrolyzed starch.

(2) Preparation of oil phase: 1000 mL of vegetable oil was weighed and added with 10 g of Tween-80 and 10 g of Span-80, and the resulting material was stirred at 60° C. for 30 min and set aside for later use.

(3) Emulsification and crosslinking: 10 g of hydrolyzed starch was dropwise added to the oil phase and stirred to react at 60° C. for 20 min, 20 mL of sodium trimetaphosphate was added to carry out crosslinking reaction for 5 h, and the reaction solution was set still after the reaction.

(4) Separation of precipitation: after the reaction solution was set still, the upper flowing liquid was removed and the lower milky white liquid was centrifuged and separated.

(6) Purification and drying: the milky body after centrifugation was washed with absolute ethanol and then subjected to suction filtration and vacuum drying at 40° C. The dried powder was sieved with a screen to obtain the product before packaging.

(7) Packaging and sterilization: the sieved product was packaged in polyethylene bottles and then plastic-sealed, and then sterilized.

### Comparative Example 4

(1) Enzymatic hydrolysis of starch: 100 g of potato starch and 10 g of amylase were added in 250 mL of phosphate buffer (pH=5), the resulting solution was stirred evenly to take a hydrolysis reaction at 45° C. for 8 h, the reaction solution was subjected to centrifugal separation at a rotating speed of 4000 r/min, the precipitation was then dried in vacuum to obtain hydrolyzed starch.

(2) Carboxymethylation of starch: 8 g of monochloroacetic acid was added in ethanol, the resulting solution was adjusted to be neutral with 0.3 mol/L sodium hydroxide solution, and then mixed with 80 g of ethanol-dissolved microporous starch prepared above; the mixture was then stirred thoroughly and transferred to a constant temperature tank; next, the mixture was continuously stirred at 50° C., 45 r/min for reaction; after 4 h, the reaction was completed, acetic acid was added to neutralize the reaction solution until the pH was 6.5, the reaction solution was then filtered, washed 3 times with ethanol, and dried for 24 h, thus obtaining carboxymethyl microporous starch.

(3) Boiling agglomeration: the above-mentioned carboxymethyl starch was placed in a boiling machine at 45° C. and added with distilled water to be coagulated and pelletized, thus obtaining a modified starch material. 50-500 μm microspheres accounted for no less than 90% of the total microsphere particles.

1. Detection of Saline Absorption Rate and Water Absorption Rate of Hemostatic Powder:

The saline absorption rate of starch was determined by natural filtration method. 0.5 g of the above hemostatic powder was weighed accurately and put into 100 mL of saline (0.9% NaCl solution) respectively. The centrifuge tube was sealed and the hemostatic powder solution was fully swelled at room temperature. The solution was then filtered with a stainless steel sieve until there was almost no water drop; then, the mass of the hemostatic powder was determined, and the water absorption rate was calculated as follows:

Q=(m₂−m₁)/m₁,

In the formula, Q is the water absorption rate, in the unit of g/g; m₁ is the mass of the hemostatic powder before water absorption, in the unit of g; m₂ is the mass of the hemostatic powder after water absorption, in the unit of g.

The water absorption rate of starch was measured by the Sessile Drop method, using the OCA40 Micro video contact angle measuring instrument from the German company Dataphysics. The results of the saline absorption rate and water absorption rate of the above-mentioned hemostatic powder are shown in Table 1.

TABLE 1
Performance comparison of hemostatic powder in saline
absorption rate and water absorption rate

| | Saline | | | |
|---|---|---|---|---|
| | absorption | Water absorption rate (mL/s) | | |
| Group | rate | First 20s | Second 20s | Third 20s |
| Example 1 | 8.3 | 0.0062 | 0.006 | 0.0055 |
| Example 2 | 9.1 | 0.0078 | 0.0075 | 0.007 |
| Example 3 | 8.2 | 0.0058 | 0.0056 | 0.0048 |
| Comparative | 8.2 | 0.0053 | 0.0052 | 0.004 |
| Example 1 | | | | |
| Comparative | 7.4 | 0.0054 | 0.005 | 0.004 |
| Example 2 | | | | |
| Comparative | 6.7 | 0.004 | 0.0035 | 0.0025 |
| Example 3 | | | | |
| Comparative | 6.5 | 0.0056 | 0.004 | 0.0032 |
| Example 4 | | | | |

The modified starch-based hemostatic powder prepared by the method of the present invention has a saline absorption rate and a water absorption rate close to or higher than that of the hemostatic powder prepared in Comparative Examples 1-4, and has a high water absorption rate and is more effective.

2. Skin Irritation Test and Sensitization Test of Hemostatic Powder

The starch-based hemostatic powder obtained in the above Examples 1-3 was tested in accordance with GB/T 14233.2-2005 and GB/T 16886.10-2005 “Biological Evaluation of Medical Devices Part 10: Stimulation and Delayed Hypersensitivity Test”. Specifically, in addition to the absorption capacity, the extraction medium (extraction medium: physiological saline and vegetable oil) was added at a ratio of 0.2 g/mL for extraction, and the test solution was prepared at (37±1°) C. for (72±2) h; the test solution was taken for test in accordance with the test method specified in GB/T 16886.10-2005.

The test sample was directly contacted with the skin on both sides of the rabbit spine for 24 h, and the gauze piece was contacted with the skin on both sides of the rabbit spine in the same way as a control. (1±0.1) h, (24±2) h, (48±2) h, and (72±2) h after the contact, erythema and edema at the contact site were scored, and the primary formic acid irritation index (PII) was scored. The test results showed that the primary irritation index (PII) of the rabbits in contact with the hemostatic powder sample was 0.0, indicating that the hemostatic powder test solutions prepared in Examples 1-3 had no skin sensitization reaction.

3. Cytotoxicity Test of Hemostatic Powder

The starch-based hemostatic powder obtained in the above Examples 1-3 was tested according to the MTT colorimetry specified in GB/T 16886.5-2016 “Biological Evaluation of Medical Devices Part 5: In Vitro Cytotoxicity Test”. Specifically, the prepared L929 fibroblast cell suspension was inoculated into a culture plate and cultured for 24 h and the supernatant was then removed. The positive control group was added with starch-based hemostatic powder test solution, the negative control group was added with negative control test solution, and the blank control group was added with fresh cell culture solution, and the culture was continued for 72 h. By observing the cell morphology and calculating the relative cell proliferation rate, the results showed that the cytotoxicity grade of the hemostatic powder prepared in Examples 1-3 was grade 1, which met the requirements for clinical use.

4. The Antibacterial Effect of Hemostatic Powder

The starch-based hemostatic powder prepared in the above Examples 1-3 was subjected to an antibacterial test, and the antibacterial effect was evaluated using the minimum inhibitory concentration MIC (mg/L). By the method of microwell dilution, 100 μL of hemostatic powder prepared in Examples 1-3 with different concentrations were added to 96 microwell plates, 10 μL of bacterial culture solution of the same concentration (10⁴ CFU/mL) was added to each microwell, incubated at 37° C. for 24 h and observed. The smallest concentration of the culture solution in the microwell without visible bacterial growth was the minimum inhibitory concentration (MIC).

TABLE 2
Comparison of the hemostatic powder solutions
of Examples 1-3 in antibacterial performance

| | MIC(mg/L) | | |
|---|---|---|---|
| Group | E. coli | S. aureus | P. aeruginosa |
| Example 1 | 15.773 | 51.352 | 62.240 |
| Example 2 | 15.987 | 63.175 | 66.002 |
| Example 3 | 14.403 | 51.093 | 61.526 |

The hemostatic powder prepared in the embodiments of the present invention has obvious antibacterial effect and low toxicity to human cells.

5. Hemostatic Effect of Hemostatic Powder on Animal Bleeding Model (Rabbit Femoral Artery Injury Model)

The test group used the starch-based hemostatic powder obtained in the above Examples 1-3. The control group did not use the starch-based hemostatic powder.

Two New Zealand albino rabbits were used in the test, and the femoral arteries of the two rabbits were punctured with a 7-gauge needle to squirt blood. After using the hemostatic powder, the test group was covered with gauze and pressed, and the control group was directly covered with gauze and pressed. The hemostatic effect was observed three minutes later.

The hemostatic powder used for the test group absorbed blood immediately after coming in contact with blood, and formed a viscous gel with blood to effectively cover the wound. It could effectively control wound bleeding within 1 minute. Moreover, after coming in contact with blood, the hemostatic powder adhered closely to the wound tissue to promote blood clotting to form a sealing effect on the damaged blood vessels of the bleeding point on the wound. The clot was not adhered to the pressing gloves or gauze dressing. When the gloves or gauze are removed, the clot was not damaged to cause secondary bleeding. The bleeding of the control group was not stopped, and the bleeding point was still bleeding after three minutes.

The above descriptions are only preferred embodiments of the present invention, and are not intended to limit the present invention in other forms. Any person familiar with the profession may use the technical content disclosed above to change or modify them into the equivalent embodiments of equivalent changes. However, any simple modifications, equivalent changes and modifications made to the above embodiments based on the technical essence of the present invention without departing from the content of the technical solutions of the present invention still belong to the protection scope of the technical solution of the present invention.

1. A preparation method of a starch-based hemostatic powder, comprising the following Steps:
(1) gelatinizing and modifying potato starch raw material to obtain modified starch;
(2) emulsifying and crosslinking the modified starch, obtained in Step (1), in an emulsifier, thus obtaining cross-linked starch; and
(3) carrying out separation, purification, drying and sterilization on the cross-linked starch obtained in Step (2), thus obtaining the starch-based hemostatic powder.

2. The preparation method according to claim 1, wherein in Step (1), the mass concentration of the starch is between 1% and 20%, the gelatinization temperature is between 40° C. and 80° C., and the gelatinization time is between 20 minutes and 120 minutes; after the gelatinization treatment, the pH is adjusted to a range from 8 to 12.

3. The preparation method according to claim 1, wherein in Step (1), a starch modifier is 2-chloroethyldiethylamine with a mass concentration between 1% and 10%, and the reaction time is between 5 h and 24 h.

4. The preparation method according to claim 1, wherein in Step (2), the emulsifier is one or more of Tween-60, Tween-80, Span-60, and Span-80.

5. The preparation method according to claim 4, wherein in Step (2), the emulsifier is first dissolved in an oil phase, heated and stirred uniformly for 10-50 minutes, and then the modified starch obtained in Step (1) is added dropwise to the solution.

6. The preparation method according to claim 5, wherein the oil phase is one of liquid paraffin, aviation kerosene, and vegetable oil; the emulsifier accounts for 0.1%-10% of the oil phase in a mass ratio, and the volume ratio of the oil phase to the aqueous solution of the starch is 3-6:1.

7. The preparation method according to claim 1, wherein in Step (2), a crosslinking agent is epichlorohydrin, and the crosslinking agent accounts for 0.2%40% of the oil phase in a mass ratio.

8. The preparation method according to claim 1, wherein in Step (2), the modified starch is emulsified for 0.5-12 h at a temperature between 35° C. and 80° C., the stirring speed of the emulsification and crosslinking process is between 100 rpm/min and 1000 rpm/min, and the crosslinking time is between 1 h and 72 h.

9. A starch-based hemostatic powder, prepared by the method according to claim 1.

10. An application of the starch-based hemostatic powder according to claim 9, wherein the starch-based hemostatic powder is used for providing one effect of hemostatic, anti-adhesion, antibacterial, and wound sealing effects on bloody wounds or their combinations; the blooding wound refers to the body surface, tissues and organs in the body, or tissues in the body cavity or organs in the body cavity of mammals, birds, and reptiles.
